(19) United States
(12) Patent Application Publication
(10) Pub. No.: US 2025/0057429 A1
(43) Pub. Date: Feb. 20, 2025

(54) CATHETER ROTARY APPARATUS FOR OPTICAL COHERENCE TOMOGRAPHY

(71) Applicants: RAYWATT INC., Seoul (KR); INDUSTRY ACADEMY COOPERATION FOUNDATION OF SEJONG UNIVERSITY, Seoul (KR)

(72) Inventors: Jin Yong HA, Seoul (KR); Sang Won WOO, Seoul (KR); Soon Won JEONG, Seoul (KR)

(21) Appl. No.: 18/617,747

(22) Filed: Mar. 27, 2024

(30) Foreign Application Priority Data
Aug. 17, 2023 (KR) 10-2023-0107893

(51) Int. Cl. A61B 5/00

(52) U.S. Cl. CPC A61B 5/0084; A61B 5/0066; A61B 2562/225

(57) ABSTRACT

A catheter rotary apparatus for OCT includes a rotary junction module and a catheter. The rotary junction module includes a body part including a through part to enable an optical fiber on a sample stage side of OCT system to pass at one end and an optical fiber on a catheter side to pass at the other end, a fiber optic rotary joint provided at one end on the sample stage side of OCT system of the body part and configured to connect the optical fiber on the sample stage side of OCT system and the optical fiber on the catheter side and a catheter. The catheter includes a female type optical connector connected to the male type optical connector, and a cap part clamped to the body part. The cap part includes an outer cap coupled to a through part formed in the body part, an inner cap inserted into the outer cap, and a fixed cap coupled to the female type optical connector and rotated when the female type optical connector is rotated.

### CROSS-REFERENCE TO RELATED APPLICATION

This application claims the benefit under 35 USC 119(a) of Korean Patent Application No. 10-2023-0107893 filed on Aug. 17, 2023 in the Korean Intellectual Property Office, the entire disclosures of which are incorporated herein by reference for all purposes.

### TECHNICAL FIELD

The present disclosure relates to a catheter rotary apparatus for optical coherence tomography (OCT).

### BACKGROUND

In general, optical coherence tomography (OCT) has been used for eye, skin and cardiovascular diseases, and provides an axial resolution of 10 μm and a lateral resolution of 20 μm to 40 μm in a direction of incident light to image internal structures of human tissues.

For OCT imaging, a semiconductor laser light source may be used to generate infrared light, and laser light (light energy) may be irradiated through a catheter (conduit) inserted in the human body so as to be incident on the surface of a target human organ. The catheter is composed of a torque coil and a nitinol tube for connecting a male type optical connector connected to a rotary junction module provided at a proximal side of the catheter. When the catheter is rotated, the torque coil and the nitinol tube are rotated together.

Then, the light reflected from the surface of the human organ returns back through the catheter. In this case, the returned light interferes with light of a reference arm. In this case, the location of a photon and various kinds of optical information about how reflection occurs can be obtained through optical interference between the two kinds of light.

In general, to obtain cardiovascular OCT images, a catheter is located in a cardiac blood vessel (with a diameter of 3 mm to 5 mm) to be examined through a femoral artery and an aorta and then captures images of the cardiac blood vessel of 54 mm in a longitudinal direction while being rotated at 180 revolutions per second and pulled back at a speed of 18 mm/s (i.e., 180 frames of the cross-sectional images of the blood vessel are played at an interval of 0.1 mm/s).

In this case, the catheter includes a torque coil and a nitinol tube which are formed as one body, and, thus, when the catheter is rotated and pulled back by a direct current motor, the left and right sides of the catheter wobble up and down. Such wobbling is worsened during high-speed rotation and transmitted to a distal side where an optical imaging lens is located, which causes image distortion. Also, in a severe case, the catheter may be in contact with the interior wall of the cardiac blood vessel, which may cause damage to the inside of the blood vessel and overall degradation in accuracy of imaging the inside of the cardiac blood vessel.

Further, when the catheter is rotated at a high speed, the motor generates vibration and thus generates noise.

### PRIOR ART DOCUMENT

### Patent Document

Korean Patent No. 10-2316478 (registered on Oct. 15, 2021)

### SUMMARY

In view of the foregoing, the present disclosure is conceived to provide a catheter rotary apparatus in which a catheter is rotated by a hollow motor provided inside a body part, a cap part clamped to the body part includes an outer cap, an inner cap, and a fixed cap, and the fixed cap is spaced apart from an inner surface of the inner cap, coupled to a rotary part and rotated when the catheter is rotated.

However, the problems to be solved by the present disclosure are not limited to the above-described problems. Although not described herein, other problems to be solved by the present disclosure can be clearly understood by a person with ordinary skill in the art from the following descriptions.

According to an aspect of the present disclosure, a catheter rotary apparatus for OCT includes a rotary junction module and a catheter. The rotary junction module includes a body part including a through part to enable an optical fiber on a sample stage side of OCT system to pass at one end and an optical fiber on a catheter side to pass at the other end, a fiber optic rotary joint provided at one end on the sample stage side of OCT system of the body part and configured to connect the optical fiber on the sample stage side of OCT system and the optical fiber on the catheter side and a catheter. The catheter includes a female type optical connector connected to the male type optical connector, and a cap part clamped to the body part. The cap part includes an outer cap coupled to a through part formed in the body part, an inner cap inserted into the outer cap, and a fixed cap coupled to the female type optical connector and rotated when the female type optical connector is rotated.

In an embodiment of the present disclosure, the rotary junction module further includes a clamp part of which one end is inserted into the through part and the other end is located to face the motor part, and the inner cap is coupled to the clamp part.

In an embodiment of the present disclosure, the rotary junction module further includes a drive part coupled to the body part and configured to move the motor part and the clamp part in one direction.

In an embodiment of the present disclosure, the clamp part includes an inner cap coupling which is inserted to be in tight contact with an inner circumferential surface of the through part and of which one end is coupled to the inner cap, and a drive part coupling coupled to the other end of the inner cap coupling and connected to the drive part.

In an embodiment of the present disclosure, the drive part includes a first drive unit connected to the motor part and configured to move the motor part in one direction, and a second drive unit connected to the clamp part and configured to move the clamp part in one direction.

In an embodiment of the present disclosure, the motor part is moved backwards in one direction by the first drive unit and the clamp part is moved backwards in one direction by the second drive unit at the same time, and, thus, the catheter is pulled back.

In an embodiment of the present disclosure, only the motor part is moved backwards in one direction by the first drive unit in a state where the clamp part has been inserted in the body part by the second drive unit, and, thus, the female type optical connector is decoupled from the male type optical connector.

In an embodiment of the present disclosure, the motor part includes a motor which has a hollow shape in order for the optical fiber on the catheter side to be inserted into the motor and connected to the male type optical connector, a fixed ring coupled to the outside of the motor and having a greater diameter than the motor, and a housing formed to enclose an outer circumferential surface of the fixed ring.

In an embodiment of the present disclosure, a plurality of grooves is recessed inwards on the outer circumferential surface of the fixed ring.

In an embodiment of the present disclosure, the motor part further includes a universal coupler which is coupled to the rotator of the fiber optic rotary joint and configured to rotate the rotator.

In an embodiment of the present disclosure, the motor part further includes an encoder coupled to one side of the motor and configured to control a rotation speed of the motor.

In an embodiment of the present disclosure, the catheter further includes a torque coil which is connected to the female type optical connector and allows insertion of the optical fiber on the catheter side and thus emits light, a first protective sheath located outside a proximal side of the torque coil, and a second protective sheath located in the entire torque coil and outside the proximal side.

In an embodiment of the present disclosure, the first protective sheath and the second protective sheath are formed of flexible materials.

In an embodiment of the present disclosure, the outer cap is rotationally coupled to the through part.

In an embodiment of the present disclosure, a plurality of catching projections is formed on an outer circumferential surface of the outer cap, a plurality of catching grooves is formed on an inner circumferential surface of the through part, and the outer cap is coupled to the through part by the plurality of catching projections and the plurality of catching grooves.

In a rotary junction module according to an embodiment of the present disclosure, a cap part includes an outer cap, an inner cap, and a fixed cap. The fixed cap is clamped to a female type optical connector and rotated when the female type optical connector is rotated. Therefore, it is possible to suppress damage to the interior wall of the cardiac blood vessel caused by excessive vibration that occurs due to wobbling of the catheter and improve accuracy of imaging the inside of the cardiac blood vessel.

However, the effects of the present disclosure are not limited to the above-described effects. Although not described herein, other effects of the present disclosure can be clearly understood by a person with ordinary skill in the art from the following descriptions.

### BRIEF DESCRIPTION OF THE DRAWINGS

Hereinafter, embodiments of the present disclosure will be described in detail with reference to the accompanying drawings so that the present disclosure may be readily implemented by a person with ordinary skill in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, it is to be understood that the term “comprises or includes” and/or “comprising or including” used in the document means that one or more other components, steps, operation and/or existence or addition of elements are not excluded in addition to the described components, steps, operation and/or elements unless context dictates otherwise and is not intended to preclude the possibility that one or more other features, numbers, steps, operations, components, parts, or combinations thereof may exist or may be added. Further, through the whole document, the term “connected to” or “coupled to” that is used to designate a connection or coupling of one element to another element includes both a case that an element is “directly connected or coupled to” another element and a case that an element is “electronically connected or coupled to” another element via still another element. Additionally, through the whole document, the term “on” that is used to designate a position of one element with respect to another element includes both a case that the one element is adjacent to the other element and a case that any other element exists between these two elements. Furthermore, the term “first,” “second,” used in the document, may denote various components and are used to qualify components irrespective of their order and/or importance, serving merely to distinguish one component from another without limiting the components themselves, and do not necessarily imply reference to different components. For example, “first direction” and “second direction” may denote the same direction or different directions.

FIG. 1 is a perspective view of a catheter rotary apparatus for OCT according to an embodiment of the present disclosure.

FIG. 2A to FIG. 2C are cross-sectional views illustrating a status when a motor part or a clamp part is moved in one direction by a drive part in the catheter rotary apparatus for OCT according to an embodiment of the present disclosure.

FIG. 3A and FIG. 3B are bottom views illustrating a status when the motor part or the clamp part is moved in one direction by the drive part in the catheter rotary apparatus for OCT according to an embodiment of the present disclosure.

FIG. 4 is a perspective view of a cap part in the catheter rotary apparatus for OCT according to an embodiment of the present disclosure.

FIG. 5 is a cross-sectional view of the cap part in the catheter rotary apparatus for OCT according to an embodiment of the present disclosure.

FIG. 6 is a diagram illustrating the motor part in the catheter rotary apparatus for OCT according to an embodiment of the present disclosure.

### DETAILED DESCRIPTION

Further, the expression “a first”, “a second”, “the first”, or “the second” used in the present disclosure may modify various components regardless of the order and/or the importance, and is used only to distinguish one element from another element, but does not limit the corresponding elements. For example, a first direction and a second direction may indicate the same direction or may indicate different directions.

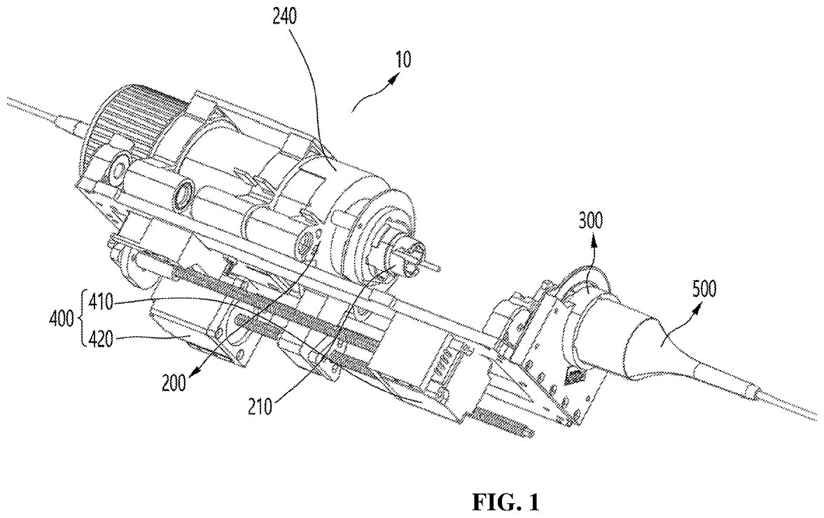

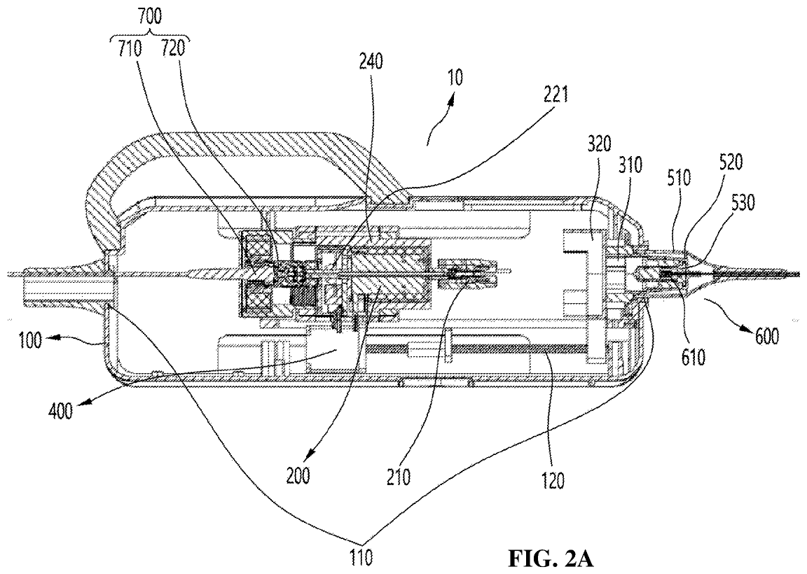

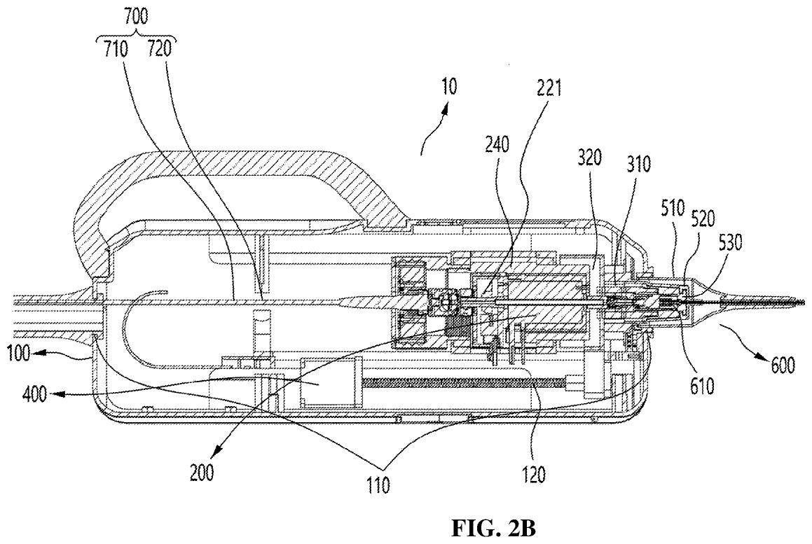

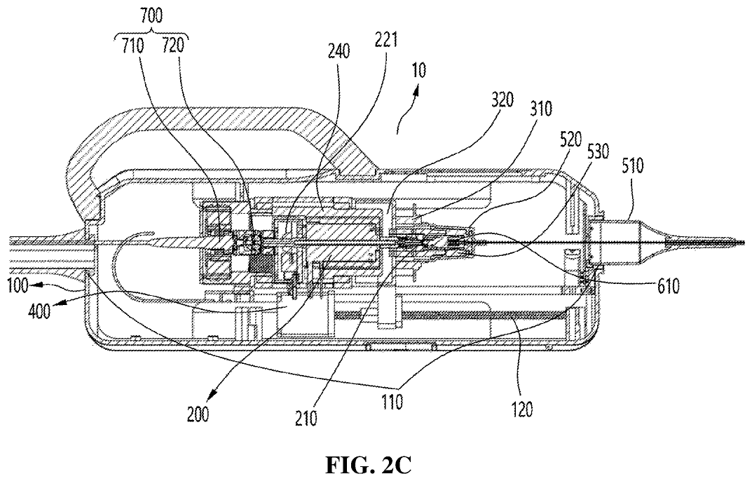

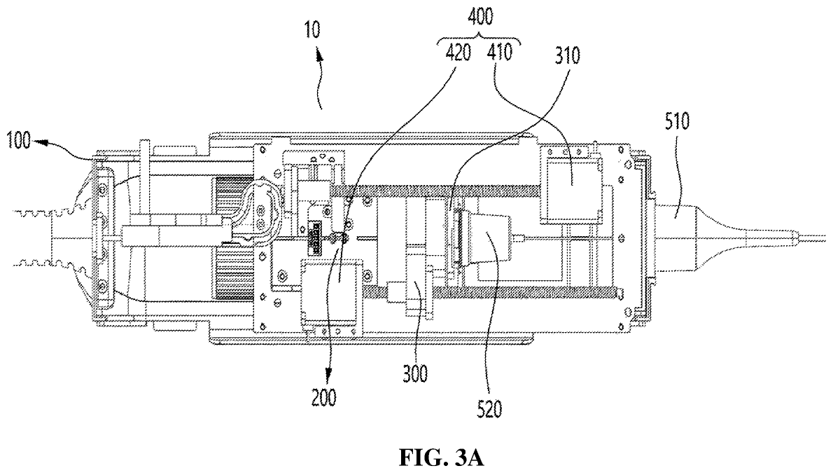

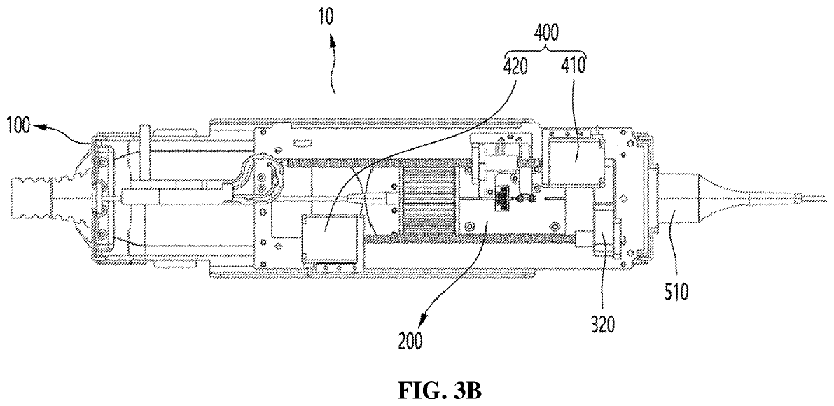

FIG. 1 is a perspective view of a catheter rotary apparatus for OCT according to an embodiment of the present disclosure, FIG. 2A is a cross-sectional view illustrating a status before a female type optical connector 210 is coupled to a catheter 600 in the catheter rotary apparatus for OCT according to an embodiment of the present disclosure, FIG. 2B is a cross-sectional view illustrating a status when a motor part 200 has been moved forward in one direction by a drive part 400 in the catheter rotary apparatus for OCT according to an embodiment of the present disclosure, FIG. 2C is a cross-sectional view illustrating a status when the motor part 200 and a clamp part 300 have been moved backwards in one direction by the drive part 400 and the catheter 600 has been pulled back in the catheter rotary apparatus for OCT according to an embodiment of the present disclosure, FIG. 3A is a bottom view illustrating a status when the motor part 200 and the clamp part 300 have been moved backwards in one direction by the drive part 400 and the catheter 600 has been pulled back in the catheter rotary apparatus for OCT according to an embodiment of the present disclosure, and FIG. 3B is a bottom view illustrating a status when the motor part 200 has been moved forward in one direction by the drive part 400 in the catheter rotary apparatus for OCT according to an embodiment of the present disclosure.

Referring to FIG. 1 to FIG. 3B, the catheter rotary apparatus for OCT according to an embodiment of the present disclosure may include a rotary junction module 10 and the catheter 600.

The rotary junction module 10 may include a body part 100, a fiber optic rotary joint 700, the motor part 200, the clamp part 300, and the drive part 400. Also, the catheter 600 may include a male type optical connector 610 and a cap part 500.

A through part 110 may be formed at one end and the other end of the body part 100 to enable an optical fiber on a sample stage side of OCT system to pass and an optical fiber on a catheter side to pass, respectively.

The through part 110 may be located corresponding to a central shaft of the motor part 200 provided inside the body part 100, and the optical fiber on the sample stage side of OCT system may pass through the through part 110 formed at one end of the body part 100 and the optical fiber on the catheter side may pass through the through part 110 formed at the other end of the body part 100. The cap part 500 may be clamped to a front end of the body part 100, and a part of the male type optical connector 610 may be inserted through the cap part 500.

A guide rail (not shown) may be provided inside the body part 100 in order for the motor part 200 to reciprocate in one direction.

The fiber optic rotary joint 700 may be provided at one end on the sample stage side of OCT system of the body part 100, and may connect the optical fiber on the sample stage side of OCT system and the optical fiber on the catheter side.

Specifically, the fiber optic rotary joint 700 may include a stator 710 connected to the optical fiber on the sample stage side of OCT system and a rotator 720 connected to the optical fiber on the catheter side passing through the inside of a shaft of the motor part 200.

When the optical fiber on the catheter side is rotated by the motor part 200, the optical fiber on the sample stage side may not be rotated through the fiber optic rotary joint 700, but may be fixed, and the fiber optic rotary joint 700 may transmit optical signals from the optical fibers in both directions between the rotator 720 and the stator 710.

The optical fiber on the sample stage side and the optical fiber on the catheter side may be overlaid with polyimide and may be single-mode fibers, but are not limited thereto. The optical fiber on the sample stage side and the optical fiber on the catheter side may be multimode fibers.

The optical fiber on the catheter side may include an optical probe coupled to an optical lens configured to emit light.

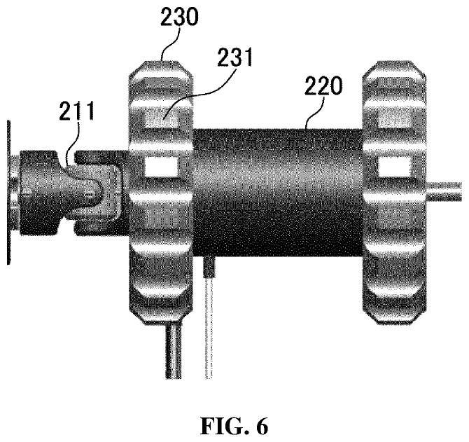

FIG. 6 is a diagram illustrating the motor part 200 in the optical rotary junction module for OCT 10 according to an embodiment of the present disclosure.

Referring to FIG. 6, the motor part 200 may be located inside the body part 100 and configured to rotate the optical fiber on the catheter side and may include the female type optical connector 210 configured to connect the optical fiber on the catheter side and the male type optical connector 610.

For example, as the female type optical connector 210 provided on one side of the motor part 200 is rotated, the motor part 200 may rotate the optical fiber on the catheter side connected to the female type optical connector 210.

The motor part 200 may include a motor 220, a fixed ring 230, and a housing 240. FIG. 6 does not illustrate the housing 240 to describe an internal structure of the motor part 200.

The motor 220 may have a hollow shape so that the optical fiber on the catheter side can be inserted into the motor 220 and connected to the female type optical connector 210.

The fixed ring 230 may be coupled to each of both ends of the motor 220, and may have a greater diameter than the motor 220.

The fixed ring 230 may be formed of a high-elasticity material, such as a silicone rubber packing, capable of absorbing vibration and noise of the motor 220, but is not limited thereto.

As shown in FIG. 6, a plurality of grooves 231 is recessed inwards on an outer circumferential surface of the fixed ring 230.

The plurality of grooves 231 may be formed at a predetermined interval and thus can minimize vibration and noise of the motor 220 transmitted to the housing 240 through the fixed ring 230.

The housing 240 may be formed to enclose the outer circumferential surface of the fixed ring 230 and thus can serve as a vibration insulating wall and sound insulating wall.

Meanwhile, the housing 240 may further include therein an encoder 221 configured to monitor a rotation speed of the motor 220.

Since the housing 240 further includes therein the encoder 221 configured to monitor the motor 220, the motor 220 can precisely control the rotation speed of the motor 220 in response to feedback from the encoder 221.

As described above, the motor part 200 includes the motor 220, the fixed ring 230, and the housing 240. Thus, it is possible to minimize transmission of vibration and noise of the motor 220 to the outside. Also, the plurality of grooves 231 is formed on the outer circumferential surface of the fixed ring 230. Thus, it is possible to further minimize transmission of vibration and noise of the motor 220 to the housing 240 through the fixed ring 230.

Meanwhile, the motor part 200 may further include a universal coupler 211 which is coupled to the rotator 720 of the fiber optic rotary joint 700 and configured to rotate the rotator 720.

According to the prior art, an optical rotary junction module is extended from an end of a motor part. In this case, the optical rotary junction module in effect causes an increase in length of a shaft of the motor part being rotated, and, thus, as a distance from the center of the motor part in an axis direction increases, wobbling is worsened by centrifugal force.

According to the present disclosure, instead of the optical rotary junction module, the universal coupler 211 is extended from an end of the motor part to enable a rotation shaft 120 of the motor part 200 to be flexibly rotated along with the rotation shaft 120 of the motor. Therefore, the motor part 200 can easily rotate the rotator 720 of the fiber optic rotary joint 700 and can also reduce the occurrence of wobbling.

For example, the universal coupler 211 may be formed of an engineering plastic material having a density of 1.1 g/cm³ to 1.5 g/cm³. When the universal coupler 211 has a density of lower than 1.1 g/cm³, its durability may decrease, and when the universal coupler 211 has a density of higher than 1.5 g/cm³, wobbling may occur and noise may increase.

Also, the universal coupler 211 may include a washer having a low coefficient of friction to suppress wear of parts of the universal coupler 211 when the universal coupler 211 is rotated at a high speed. For example, the washer of the universal coupler 211 may have a coefficient of friction of 0.05μ to 0.13μ.

Since the motor part 200 and the clamp part 300 to be described later reciprocate in one direction by means of the drive part 400, the cap part 500 can be clamped to the body part 100 and the male type optical connector 610 can be engaged with and disengaged from the female type optical connector 210.

The drive part 400 may include a first drive unit 410 configured to move the motor part 200 in one direction and a second drive unit 420 configured to move the clamp part 300 in one direction.

As shown in FIG. 1 to FIG. 3B, the first drive unit 410 is provided at a lower portion of the body part 100, and the motor part 200 is connected to a first rotation shaft 120 included in the first drive unit 410. Thus, when the first drive unit 410 is operated, the motor part 200 can be moved forward or moved backwards in one direction.

As shown in FIG. 1 to FIG. 3B, the second drive unit 420 is provided at the lower portion of the body part 100, and the clamp part 300 is connected to a second rotation shaft 120 included in the second drive unit 420. Thus, when the second drive unit 420 is operated, the clamp part 300 can be moved forward or moved backwards in one direction.

In this case, as shown in FIG. 1 to FIG. 3B, the first drive unit 410 and the second drive unit 420 may be respectively provided at both lower portions of the body part 100 to avoid spatial constraints caused by the size of the drive units 410 and 420.

As shown in FIG. 1 to FIG. 3B, the drive part 410 may be provided at the lower portion of the body part 100 and driven such that the motor part 200 reciprocates along the guide rail (not shown) provided inside the body part 100.

As shown in FIG. 1 to FIG. 3B, the clamp part 300 may be inserted into the through part 110, and, thus, the other end of the clamp part 300 may be located to face the motor part 200.

The clamp part 300 may be moved in one direction along the guide rail (not shown) by the drive part 420.

The clamp part 300 may include an inner cap coupling 310 and a drive part coupling 320.

As shown in FIG. 2A to FIG. 3B, the inner cap coupling 310 may be inserted to be in tight contact with an inner circumferential surface of the through part 110 and one end of the inner cap coupling 310 may be coupled to an inner cap 520 to be described later. Thus, it is possible to suppress separation of the male type optical connector 610 to the outside.

One end of the drive part coupling 320 may be coupled to the other end of the inner cap coupling 310 and the other end may be connected to the drive part 400.

For example, the drive part coupling 320 may be connected to the rotation shaft 120 included in the drive part 420 so that the clamp part 300 can be moved forward or moved backwards in one direction when the drive part 420 is operated.

The drive part 420 may be coupled to an upper portion of the body part 100 and thus can move the clamp part 300 in one direction.

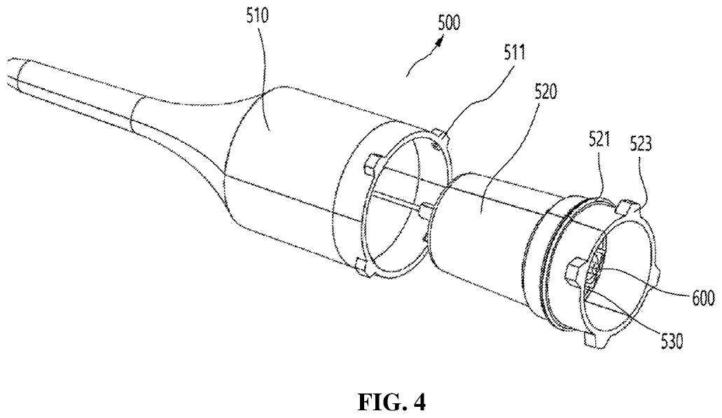

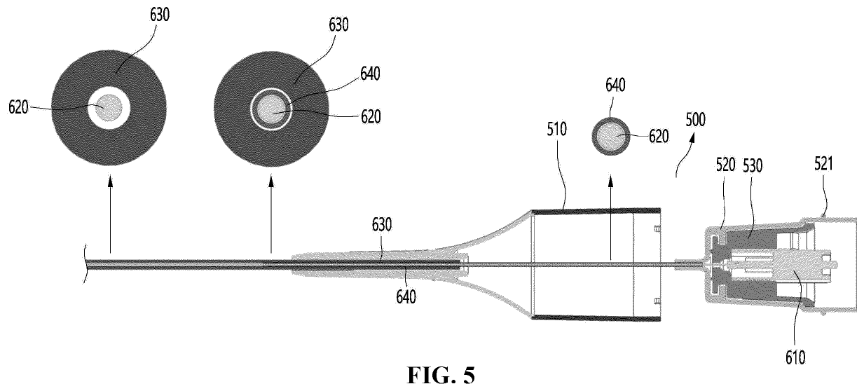

FIG. 4 is a perspective view of the cap part 500 in the catheter rotary apparatus for OCT according to an embodiment of the present disclosure, and FIG. 5 is a cross-sectional view of the cap part 500 in the catheter rotary apparatus for OCT according to an embodiment of the present disclosure.

The male type optical connector 610 included in the catheter 600 may be inserted into the cap part 500, and the cap part 500 may be clamped to the body part 100.

The cap part 500 may be clamped to the body part 100 in a state where the male type optical connector 610 and a torque coil 620 to be described later have been inserted in the cap part 500.

Specifically, as shown in FIG. 4 and FIG. 5, the cap part 500 may include an outer cap 510, the inner cap 520, and a fixed cap 530.

The outer cap 510 may be rotationally coupled to the through part 110 formed in the body part 100.

For example, as shown in FIG. 1 to FIG. 5, one end of the outer cap 510 extends convergently in one direction and has a diameter equivalent to that of the torque coil 620 of the catheter 600, and the other end of the outer cap 510 may have a diameter with which the inner cap coupling 310 inserted in the through part 110 of the body part 100 can be inserted into the outer cap 510.

The outer cap 510 may include therein a space that enables insertion of the inner cap 520. For example, when the inner cap 520 is inserted into the outer cap 510, the inner cap 520 may be spaced apart from the outer cap 510.

As the outer cap 510 is rotationally coupled to the through part 110 formed in the body part 100, the motor part 200 is moved forward in one direction by the drive part 410. Thus, when the male type optical connector 610 is in contact with the cap part 500, it is possible to suppress separation of the outer cap 510 from the through part 110.

Alternatively, the outer cap 510 may be coupled to the through part 110 by a catching projection 511 and a catching groove.

For example, a plurality of catching projections 511 may be formed on an outer circumferential surface of the outer cap 510, and a plurality of catching grooves (not shown) may be formed on an inner circumferential surface of the through part 110. Thus, when a part of the outer cap 510 together with the inner cap 520 is inserted into the through part 110, the plurality of catching projections 511 of the outer cap 510 can be coupled and fixed to the plurality of catching grooves (not shown) of the through part 110.

Meanwhile, the outer cap 510 and the inner cap 520 may be clamped together at the same time, or the outer cap 510 may be clamped after the inner cap 520 is clamped.

The inner cap 520 may be inserted into the outer cap 510 and coupled to the clamp part 300.

For example, the inner cap 520 may include a catching part 521 protruding outwards along an outer edge so that only a part of the inner cap 520 can be inserted into the clamp part 300.

For example, the catching part 521 protruding outwards along the outer edge may be formed on a stop surface of the inner cap 520 and the motor part 200 may be moved forward in one direction by the drive part 400 to be in contact with the cap part 500, and, thus, only a part of the inner cap 520 can be inserted into the clamp part 300 through the catching part 521.

Also, when a part of the inner cap 520 is inserted into the outer cap 510, a plurality of catching projections 523 may be formed on an outer circumferential surface of the inserted inner cap 520 and coupled and fixed to catching grooves (not shown) recessed inwards on the catching projections 511 of the outer cap 510. Therefore, the inner cap 520 can be moved together when the motor part 200 is moved backwards (pulled back) in one direction by the drive part 400.

Meanwhile, unlike the fixed cap 530 to be described later, the inner cap 520 is not rotated together with the male type optical connector 610 when the male type optical connector 610 is rotated by the motor part 200.

Herein, the female type optical connector 210, the fixed cap 530, the male type optical connector 610, and the torque coil 620 may form a rotary part, and the rotary part may be rotated by the motor part 200. Meanwhile, the inner cap 520 and the outer cap 510 may form a fixed part, and the fixed part is not rotated when the rotary part is rotated.

As described above, the inner cap 520 is coupled to the clamp part 300, and, thus, it is possible to suppress separation of the rotary part to the outside. Also, the inner cap 520 is in contact with one end of the fixed cap 530, and, thus, it is possible to further reduce wobbling of the rotary part.

The fixed cap 530 is spaced apart from an inner surface of the inner cap 520, clamped to the male type optical connector 610, and rotated when the male type optical connector 610 is rotated.

For example, as shown in FIG. 4 and FIG. 5, one end of the fixed cap 530 may be in contact with the inside of the inner cap 520, and an outer surface of the fixed cap 530 may be spaced apart from the inner surface of the inner cap 520 to have a tolerance therebetween.

The torque coil 620 connected to the male type optical connector 610 may be inserted into the fixed cap 530, and the male type optical connector 610 may be clamped and fixed to an inner circumferential surface of the fixed cap 530.

As shown in FIG. 2A and FIG. 2B, the female type optical connector 210 may be coupled to the motor part 200 when the motor part 200 is moved forward in one direction by the first drive unit 410. In this case, the female type optical connector 210 may be coupled to the motor part 200 by various coupling methods such as a hook coupling method, a bolt coupling method, etc.

As described above, the fixed cap 530 is spaced apart from the inner surface of the inner cap 520, coupled to the male type optical connector 610, and rotated when the rotary part is rotated. Thus, it is possible to reduce wobbling occurring in the torque coil 620 of the rotary part.

Meanwhile, the catheter 600 may be a disposable component configured to be inserted into a cylindrical part, such as the inside of the blood vessel, throat, large intestine, etc., of a human body and to emit light. The catheter 600 may be inserted into the inside of the blood vessel of a human body and used to image information about the inside of the blood vessel.

The catheter 600 may function to emit light, but may not function to generate light by itself. Therefore, the catheter 600 may be clamped to an optical fiber connected to the OCT system through the female type optical connector 210 and supplied with light.

In a conventional catheter, a nitinol tube is used between a female type optical connector and a torque coil to reduce up-and-down wobbling of the left and right sides of the catheter when the catheter is rotated by a motor. However, the catheter has a low wobbling reduction efficiency and is costly due to the nitinol tube.

The catheter 600 used in the catheter rotary apparatus for OCT according to an embodiment of the present disclosure does not use a nitinol tube which has been used in a conventional catheter. Thus, it is possible to reduce the cost of the catheter 600.

The catheter 600 may further include the torque coil 620, a first protective sheath 630, and a second protective sheath 640.

The torque coil 620 may be connected to the male type optical connector 610 and allows insertion of the optical fiber on the catheter side and thus can emit light.

The torque coil 620 is formed into several layers of a multi-coil configuration that can be rotated at a high speed in situ in the blood vessel under precise control and thus can be improved in flexibility and torque transmission. The first protective sheath 630 may be located outside a proximal side of the torque coil 620 and fixed by the inner cap 520. When the motor part 200 is rotated, only the torque coil 620 constituting the rotary part is rotated, but the first protective sheath 630 is not rotated.

For example, the first protective sheath 630 may have a thickness sufficient for the torque coil 620 to be inserted into the first protective sheath 630 and not to cause friction when the torque coil 620 is rotated by motor part 200, but is not limited thereto. The first protective sheath 630 may have a thickness equal to or similar to that of the nitinol tube used in the conventional catheter. Also, the first protective sheath 630 may have a greater length than a moving distance when the catheter 600 is pulled back. For example, when a pullback distance is 100 mm, the first protective sheath 630 may have a length of 110 mm to 150 mm, preferably 110 mm to 120 mm.

Since the first protective sheath 630 is located outside the proximal side of the torque coil 620 and fixed by the inner cap 520 (which is not rotated as described above), it is possible to minimize wobbling and vibration of the torque coil 620 included in the rotary part when the rotary part is rotated by the motor part 200.

The first protective sheath 630 may be formed of flexible materials, such as nylon and polyimide.

The second protective sheath 640 may be located outside the entire torque coil 620 (as well as outside the first protective sheath 630).

For example, as shown in FIG. 5, the second protective sheath 640 may be connected to the outer cap 510 and the first protective sheath 630 may be inserted into the second protective sheath 640.

In this case, an inner circumferential surface of the second protective sheath 640 may be spaced apart from an outer circumferential surface of the first protective sheath 630, but the present disclosure is not limited thereto. The inner circumferential surface of the second protective sheath 640 may be in tight contact with the outer circumferential surface of the first protective sheath 630.

Since the second protective sheath 640 is connected to one end of the outer cap 510 and located in the entire torque coil 620 and outside the proximal side, it is possible to further minimize wobbling and vibration of the torque coil 620 included in the rotary part when the rotary part is rotated by the motor part 200.

Like the first protective sheath 630, the second protective sheath 640 may be formed of flexible materials, such as nylon.

As described above, in the catheter rotary apparatus according to an embodiment of the present disclosure, the catheter 600 further includes the first protective sheath 630 and the second protective sheath 640. Thus, it is possible to minimize wobbling and vibration of the torque coil 620 included in the rotary part when the rotary part is rotated by the motor part 200.

That is, the catheter rotary apparatus according to an embodiment of the present disclosure uses the inner cap 520, the first protective sheath 630, and the second protective sheath 640 to solve wobbling and vibration of a conventional nitinol tube when capturing images of the blood vessel.

Meanwhile, in the rotary junction module 10, the motor part 200 may be moved backwards in one direction by the first drive unit 410 and the clamp part 300 may be moved backwards in one direction by the second drive unit 420 at the same time, and, thus, the rotary part may be pulled back.

Herein, the term “pullback” refers to a process of locating the torque coil 620 inside the blood vessel and then pulling the torque coil 620 in one side to capture images of the blood vessel. As the motor part 200 and the clamp part 300 are moved backwards in one direction by the drive part 400, the female type optical connector 210, the fixed cap 530, the male type optical connector 610, and the torque coil 620 included in the rotary part may be moved backwards in one direction and the inner cap 520 coupled to the clamp part 300 may also be moved backwards in one direction.

For example, as shown in FIG. 2B and FIG. 2C, when the male type optical connector 610 is coupled to the female type optical connector 210 as the motor part 200 is moved forward in one direction by the first drive unit 410, the motor part 200 and the clamp part 300 may be moved backwards in one direction, and, thus, the rotary part may be pulled back.

In this case, the outer cap 510 may be coupled to the through part 110 and fixed to the body part 100. Also, the inner cap 520 and the fixed cap 530 may be coupled to the inner cap coupling 310 of the clamp part 300. Therefore, when the rotary part is pulled back, the inner cap 520 may also be moved backwards.

When the male type optical connector 610 is decoupled from the female type optical connector 210, only the motor part 200 may be moved backwards in one direction by the first drive unit 410 in a state where the clamp part 300 has been inserted in the body part 100 by the second drive unit 420, as shown in FIG. 2A and FIG. 2B.

In this case, the clamp part 300 inserted in the body part 100 is fixed, and, thus, it is possible to suppress movement of the inner cap 520 and the fixed cap 530 into the body part 100 together with the female type optical connector 210. Also, only the motor part 200 is moved backwards in one direction, and, thus, it is possible to decouple the male type optical connector 610 from the female type optical connector 210.

As described above, in the catheter rotary apparatus for OCT according to an embodiment of the present disclosure, the cap part 500 includes the outer cap 510, the inner cap 520, and the fixed cap 530. Also, the fixed cap 530 is spaced apart from the inner surface of the inner cap 520, clamped to the male type optical connector 610 and rotated when the male type optical connector 610 is rotated and thus reduces wobbling which occurs in the catheter 600. Therefore, it is possible to suppress a contact with the interior wall of the cardiac blood vessel and improve accuracy of imaging the inside of the cardiac blood vessel.

Further, the motor part 200 includes the motor 220, the fixed ring 230, and the housing 240. Furthermore, the fixed ring 230 having a greater diameter than the motor 220 is coupled to the outside of the motor 220, and the housing 240 is formed to enclose the outer circumferential surface of the fixed ring 230. Thus, it is possible to reduce noise caused by vibration of the motor 220.

Moreover, the catheter 600 used in the catheter rotary apparatus for OCT is composed of the male type optical connector 610 and the torque coil 620. Thus, it is possible to reduce the cost of the catheter 600.

Besides, the catheter 600 further includes the first protective sheath 630 and the second protective sheath 640. Thus, it is possible to minimize wobbling and vibration of the torque coil 620 included in the rotary part when the rotary part is rotated by the motor part 200.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure.

The scope of the present disclosure is defined by the following claims rather than by the detailed description of the embodiment. It shall be understood that all modifications and embodiments conceived from the meaning and scope of the claims and their equivalents are included in the scope of the present disclosure.

### EXPLANATION OF CODES

10: Rotary junction module
100: Body part
110: Through part
120: Rotation shaft
200: Motor part
210: Female type optical connector
211: Universal coupler
220: Motor
230: Fixed ring
231: Groove
240: Housing
300: Clamp part
310: Inner cap coupling
320: Drive part coupling
400: Drive part
410: First drive unit
420: Second drive unit
500: Cap part
510: Outer part
520: Inner cap
521: Catching part
523: Catching projection
530: Fixed cap
600: Catheter
610: Male type optical connector
620: Torque coil
630: First protective sheath
640: Second protective sheath
700: Fiber optic rotary joint
710: Stator
720: Rotator

1. A catheter rotary apparatus for OCT, comprising:
a rotary junction module which includes
a body part including a through part to enable an optical fiber on a sample stage side of OCT system to pass at one end and an optical fiber on a catheter side to pass at the other end,
a fiber optic rotary joint provided at one end on the sample stage side of OCT system of the body part and configured to connect the optical fiber on the sample stage side of OCT system and the optical fiber on the catheter side, and
a motor part which is provided inside the body part and configured to rotate the optical fiber on the catheter side, and includes a male type optical connector configured to connect the optical fiber on the catheter side and a catheter; and
a catheter which includes
a female type optical connector connected to the male type optical connector, and
a cap part clamped to the body part,
wherein the cap part includes:
an outer cap coupled to a through part formed in the body part;
an inner cap inserted into the outer cap; and
a fixed cap coupled to the female type optical connector and rotated when the female type optical connector is rotated.

2. The catheter rotary apparatus of claim 1,
wherein the rotary junction module further includes
a clamp part of which one end is inserted into the through part and the other end is located to face the motor part, and
the inner cap is coupled to the clamp part.

3. The catheter rotary apparatus of claim 2,
wherein the rotary junction module further includes
a drive part coupled to the body part and configured to move the motor part and the clamp part in one direction.

4. The catheter rotary apparatus of claim 3,
wherein the clamp part includes:
an inner cap coupling which is inserted to be in tight contact with an inner circumferential surface of the through part and of which one end is coupled to the inner cap; and
a drive part coupling coupled to the other end of the inner cap coupling and connected to the drive part.

5. The catheter rotary apparatus of claim 3,
wherein the drive part includes:
a first drive unit connected to the motor part and configured to move the motor part in one direction; and
a second drive unit connected to the clamp part and configured to move the clamp part in one direction.

6. The catheter rotary apparatus of claim 5,
wherein the motor part is moved backwards in one direction by the first drive unit and the clamp part is moved backwards in one direction by the second drive unit at the same time, and, thus, the catheter is pulled back.

7. The catheter rotary apparatus of claim 5,
wherein only the motor part is moved backwards in one direction by the first drive unit in a state where the clamp part has been inserted in the body part by the second drive unit, and, thus, the female type optical connector is decoupled from the male type optical connector.

8. The catheter rotary apparatus of claim 1,
wherein the motor part includes:
a motor which has a hollow shape in order for the optical fiber on the catheter side to be inserted into the motor and connected to the male type optical connector;
a fixed ring coupled to the outside of the motor and having a greater diameter than the motor; and
a housing formed to enclose an outer circumferential surface of the fixed ring.

9. The catheter rotary apparatus of claim 8,
wherein a plurality of grooves is recessed inwards on the outer circumferential surface of the fixed ring.

10. The catheter rotary apparatus of claim 1,
wherein the motor part further includes a universal coupler which is coupled to the rotator of the fiber optic rotary joint and configured to rotate the rotator.

11. The catheter rotary apparatus of claim 8,
wherein the motor part further includes
an encoder coupled to one side of the motor and configured to control a rotation speed of the motor.

12. The catheter rotary apparatus of claim 1,
wherein the catheter further includes:
a torque coil which is connected to the female type optical connector and allows insertion of the optical fiber on the catheter side and thus emits light;
a first protective sheath located outside a proximal side of the torque coil; and
a second protective sheath located in the entire torque coil and outside the proximal side.

13. The catheter rotary apparatus of claim 12,
wherein the first protective sheath and the second protective sheath are formed of flexible materials.

14. The catheter rotary apparatus of claim 1,
wherein the outer cap is rotationally coupled to the through part.

15. The cart catheter rotary apparatus of claim 1,
wherein a plurality of catching projections is formed on an outer circumferential surface of the outer cap, a plurality of catching grooves is formed on an inner circumferential surface of the through part, and the outer cap is coupled to the through part by the plurality of catching projections and the plurality of catching grooves.
